(19) United States
(12) Patent Application Publication
(10) Pub. No.: US 2009/0312356 A1
(43) Pub. Date: Dec. 17, 2009

(54) NICOTINIC AGONISTS SELECTIVE FOR THE ALPHA7 RECEPTOR SUBTYPE, THE PROCESS FOR THE PREPARATION THEREOF AND PHARMACEUTICAL COMPOSITIONS THEREFROM

(75) Inventors: Carlo De Micheli, Pavia (IT); Marco De Amici, Pavia (IT); Clelia Dallanoce, Milano (IT); Francesco Clementi, Milano (IT); Cecilla Gotti, Milano (IT)

Correspondence Address: Steinfl & Bruno, 301 N Lake Ave Ste 810, Pasadena, CA, 91101, US

(73) Assignees: UNIVERSITA DEGLI STUDI DI MILANO, Milano (IT); CONSIGLIO NAZIONALE DELLE RICERCHE, Roma (IT)

(21) Appl. No.: 12/306,523

(22) PCT Filed: Jun. 28, 2007
(86) PCT No.: PCT/EP2007/005724

§ 371 Date: Aug. 19, 2009

(30) Foreign Application Priority Data
Jun. 30, 2006 (IT) MI2006A001279

(51) Int. Cl. A61K 31/438; C07D 471/10; A61P 25/28; A61P 25/22; A61P 25/34; A61P 25/18; A61P 25/24; A61P 25/16

(52) U.S. Cl. 514/278; 546/18

(57) ABSTRACT

The invention discloses compounds of formula I endowed with agonistic activity at the alpha7 nicotinic acetylcholine receptors (α7 nAChRs), a process for the preparation thereof, pharmaceutical compositions containing the same and the use thereof for the treatment of neurological and psychiatric disorders as well as inflammatory diseases.

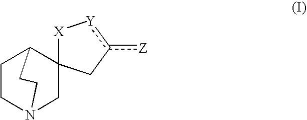

The present invention relates to compounds endowed with agonistic activity at the alpha7 nicotinic acetylcholine receptors (α7 nAChRs), a process for their preparation, pharmaceutical compositions containing the same and the use thereof for the treatment of neurological and psychiatric disorders as well as inflammatory diseases.

### BACKGROUND OF THE INVENTION

Neuronal nicotinic acetylcholine receptors (nAChRs) make up a family of pentameric ligand-gated ion channels which are formed by combinations of alpha and beta subunits¹ or existing as homopentamers, in the cases of α7, α8, and α9 receptors, which are inhibited by α-bungarotoxin.² To date, nine α and three β isoforms have been discovered, though only a relatively small subset of combinations generates functionally and physiologically relevant channels.³ Nicotinic receptors are widely distributed in the human brain, where they are frequently associated with modulatory events and, to a lesser extent, mediate synaptic transmission.⁴ The homomeric α7 subtype is highly permeable to calcium and has been proposed to be involved in the regulation of attentional as well as cognitive processes.^{2,5} In particular, these receptors are highly expressed in the brain cortex, in the subcortical, limbic regions, and the hippocampus, where they modulate inhibitory GABAergic synaptic transmission involved in sensory processing.^{6,7} Deficits in auditory sensory processing are thought to lead to a state of sensory overload and are hypothesized to contribute to the attentional and cognitive dysfunctions in a number of central nervous system diseases, among them schizophrenia.^{8,9} Moreover, intracerebroventricular injections of α-bungarotoxin (α-BTX) and α7 (and α8, α9) receptor antagonists disrupt hippocampal auditory gating.⁷ Further connections between α7 receptors and some aspects of schizophrenia reside in the observation of decreased levels of this receptor in the postmortem brains of schizophrenic patients.^{10,11} As a consequence, the α7 subtype has been the most intensively studied nAChR in recent years and a growing number of patent applications for α7 nAChR ligands, allosteric modulators and uses thereof demonstrate interest in view of a promising therapeutic application.¹²⁻¹⁵ The advancement of some of these agents into preclinical (SSR 180711, Sanofi-Aventis)^{16,17} and clinical (e.g., PH-399733, Pfizer; MEM 3454, Memory Pharmaceuticals/Roche) trials confirm the interest in the development of novel compounds selectively acting at this receptor subtype for an innovative treatment of neurological and psychiatric pathologies. Moreover, since recent reports evidenced a role of α7 nAChRs as essential regulators of inflammation,^{18,19} full agonists of this receptor subtype might find an application in the treatment of inflammatory diseases.^{20,21}

### BRIEF SUMMARY OF THE INVENTION

The invention provides compounds selectively acting as full or partial agonists at the α7 nAChRs, the procedure for their synthesis, pharmaceutical compositions containing such compounds and the use thereof for the treatment of pathologies which may benefit from the activation of the α7 nAChRs, e.g. neurological and psychiatric disorders such as Alzheimer's disease and schizophrenia and inflammatory processes.

### DISCLOSURE OF THE INVENTION

Compounds of the invention are ligands for nicotinic acetylcholine receptors (nAChRs) of formula I:

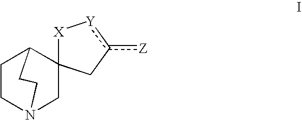

and pharmaceutically acceptable salts or enantiomer thereof, wherein:

a) when X is oxygen and Y is nitrogen, then, in the Y═C-Z moiety,

Z is selected from halogen; hydrogen; linear, branched or cyclic (C₁-C₆) alkyl, haloalkyl, alkoxy or acyl; (C₂-C₆) alkenyl, alkenyloxy; (C₂-C₆) alkynyl, alkynyloxy; benzyl, benzyloxy, (Ar) aryl, aryloxy; hydroxy; hydroxymethyl; cyano; nitro; amino; mono- or di-(C₁-C₆) alkylamino, aminomethyl, alkylaminomethyl, acylamino, alkylaminocarbonyl groups; linear, branched or cyclic (C₁-C₆) alkoxy-, (C₂-C₆) alkenyloxy-, (C₂-C₆) alkynyloxy- or (Ar) aryloxycarbonyl groups;

Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar moiety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl

with the proviso that Z is not methyl, tert-butyl, phenyl or 2,4,6-trimethylphenyl;

b) when X is a group NR,

R is selected from hydrogen, linear, branched or cyclic (C₁-C₆) alkyl, benzyl, (Ar) aryl;

Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, -5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar moiety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl

and Y is nitrogen, then, in the Y═C-Z moiety,

Z is selected from halogen; hydrogen; linear, branched or cyclic (C₁-C₆) alkyl, haloalkyl, alkoxy or acyl; (C₂-C₆) alkenyl, alkenyloxy; (C₂-C₆) alkynyl, alkynyloxy; benzyl, benzyloxy, (Ar) aryl, aryloxy; hydroxy; hydroxymethyl; cyano; nitro; mono- or di-(C₁-C₆) alkylamino, aminomethyl, alkylaminomethyl, acylamino, alkylaminocarbonyl groups; linear, branched or cyclic (C₁-C₆) alkoxy-, (C₂-C₆) alkenyloxy-, (C₂-C₆) alkynyloxy- or aryloxy-carbonyl groups;

Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar moiety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C,-C₆) alkylamino, aminomethyl

c) when X is oxygen and Y is a group NR

R is selected from hydrogen; linear, branched or cyclic (C₁-C₆) alkyl, (C₂-C₆) alkenyl, (C₂-C₆) alkynyl; benzyl; (Ar) aryl Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar moiety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl

then, in the Y—C=Z moiety, Z is oxygen.

The compounds disclaimed under a) are known from Arkivoc, 2006 (iii), 175-183 wherein they are reported to be inactive as acetylcholinesterase inhibitors. These compounds may however be used as agonists at the α7 nAChRs according to the invention.

According to a first embodiment, the invention provides compounds of formula Ia

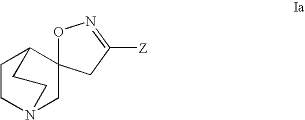

wherein Z is selected from Br, Cl; H; C₂H₅, n-C₃H₇, CH(CH₃)₂, n-C₄H₉, CH₂CH(CH₃)₂, OCH₃, OC₂H₅, O-n-C₃H₇, OCH(CH₃)₂, O-n-C₄H₉, OCH₂CH(CH₃)₂, OC(CH₃)₃; CH═CH₂, CH₂—CH═CH₂, OCH═CH₂, OCH₂—CH═CH₂; C≡CH, CH₂—C≡CH, C₂H₄—C≡CH, CH₂—C≡C—CH₃, OC≡CH, OCH₂—C≡CH, OC₂H₄—C≡CH, OCH₂—C≡C—CH₃; CH₂—C₆H₅, OCH₂—C₆H₅; 4-phenyl, 2-pyridyl, 3-pyridyl, 4-pyridyl; 2-pyrimidyl, 4-pyrimidyl, 5-pyrimidyl; 2-pyrazinyl; 2-furyl, 3-furyl; 2-thiophenyl, 3-thiophenyl; 2-pyrrolyl, 3-pyrrolyl, OC₆H₅, O-4-chloro-phenyl, O-2,4,6-trimethyl-phenyl, O-2-pyridyl, O-3-pyridyl, O-4-pyridyl; O-2-pyrimidyl, O-4-pyrimidyl, O-5-pyrimidyl; O-2-pyrazinyl, O-3-pyrazinyl; O-2-furyl, O-3-furyl; O-2-thiophenyl, O-3-thiophenyl; O-2-pyrrolyl, O-3-pyrrolyl; OH, CH₂OH; CH₂NH₂; CN; COOCH₃, COOC₂H₅, COOPh, COOCH₂Ph.

According to a second embodiment, the invention provides compounds of formula Ib

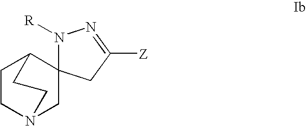

wherein:

R is selected from H, CH₃, C₂H₅, n-C₃H₇, CH(CH₃)₂, CH₂—C₆H₅, phenyl, 2,4,6-trimethyl-phenyl, 4-chloro-phenyl; 2-pyridyl, 3-pyridyl, 4-pyridyl, 2-thiophenyl, 3-thiophenyl,

and Z is selected from Br, Cl; H; CH₃, C₂H₅, n-C₃H₇, CH(CH₃)₂, n-C₄H₉, CH₂CH(CH₃)₂, C(CH₃)₃, OCH₃, OC₂H₅, O-n-C₃H₇, OCH(CH₃)₂, O-n-C₄H₉, OCH₂CH(CH₃)₂, OC(CH₃)₃; CH═CH₂, CH₂—CH═CH₂, OCH═CH₂, OCH₂—CH═CH₂; C≡CH, CH₂—C≡CH, C₂H₄—C≡CH, CH₂—C≡C—CH₃, OC≡CH, OCH₂—C≡CH, OC₂H₄—C≡CH, OCH₂—C≡C—CH₃; CH₂—C₆H₅, OCH₂—C₆H₅; phenyl, 2,4,6-trimethyl-phenyl, 4-chloro-phenyl, 2-pyridyl, 3-pyridyl, 4-pyridyl; 2-pyrimidyl, 4-pyrimidyl, 5-pyrimidyl; 2-pyrazinyl, 2-furyl, 3-furyl; 2-thiophenyl, 3-thiophenyl; 2-pyrrolyl, 3-pyrrolyl, OC₆H₅, O-4-chloro-phenyl, O-2,4,6-trimethyl-phenyl, O-2-pyridyl, O-3-pyridyl, O-4-pyridyl; O-2-pyrimidyl, O-4-pyrimidyl, O-5-pyrimidyl; O-2-pyrazinyl, O-3-pyrazinyl; O-2-furyl, O-3-furyl; O-2-thiophenyl, O-3-thiophenyl; O-2-pyrrolyl, O-3-pyrrolyl; OH, CH₂OH; CH₂NH₂; CN; COOCH₃, COOC₂H₅, COOPh, COOCH₂Ph.

According to a further embodiment, the invention provides compounds of formula Ic

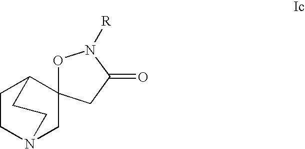

wherein R is selected from H, CH₃, C₂H₅, n-C₃H₇, CH(CH₃)₂, n-C₄H₉, CH₂CH(CH₃)₂, C(CH₃)₃; CH₂—CH═CH₂; CH₂—C≡CH, C₂H₄—C≡CH; phenyl, 2,4,6-trimethyl-phenyl, 4-chloro-phenyl, CH₂—C₆H₅; 2-pyridyl, 3-pyridyl, 4-pyridyl, 2-thiophenyl, 3-thiophenyl.

In a particular aspect the invention relates to the use of compounds according to formula I for the therapy of diseases mediated through the action of nicotinic acetylcholine receptors. A more particular aspect of the invention relates to the use of compounds of formula I for the therapy of diseases mediated through the action of α7 nicotinic acetylcholine receptors.

Another aspect of the invention relates to a method of treatment or prophylaxis of human diseases or conditions in which activation of the α7 nicotinic receptor is beneficial which comprises administering a therapeutically effective amount of a compound of the invention.

Another aspect of the invention relates to a method of treatment or prophylaxis of neurological disorders, psychotic disorders or intellectual impairment disorders, which comprises administering a therapeutically effective amount of a compound of the invention.

Another aspect of the invention relates to a method of treatment or prophylaxis, wherein the disorder is Alzheimer's disease, learning deficit, cognition deficit, attention deficit, memory loss or Attention Deficit Hyperactivity Disorder.

Another aspect of the invention relates to a method of treatment or prophylaxis, wherein the disorder is Parkinson's disease, Huntington's disease, Tourette's syndrome or neurodegenerative disorders in which there is loss of cholinergic synapses.

Another aspect of the invention relates to a method of treatment or prophylaxis, wherein the disorder is anxiety, schizophrenia or mania or manic depression.

Another aspect of the invention relates to a method of treatment or prophylaxis, wherein the disorders are inflammatory diseases.

Another aspect of the invention relates to a method of treatment or prophylaxis of jetlag, cessation of smoking, nicotine addiction, craving, pain, and ulcerative colitis, which comprises administering a therapeutically effective amount of a compound of the invention.

Another aspect of the invention relates to a pharmaceutical composition comprising a compound of the invention and a pharmaceutically acceptable diluent or carrier.

Another aspect of the invention relates to the use of a compound of the invention in the manufacture of a medicament for treatment or prophylaxis of inflammatory diseases.

Another aspect of the invention relates to the use of a compound as described above in the manufacture of a medicament for the treatment or prophylaxis of jetlag, pain or ulcerative colitis.

Another aspect of the invention relates to the use of a compound of the invention in the manufacture of a medicament for facilitating the cessation of smoking or the treatment of nicotine addiction or craving including that resulting from exposure to products containing nicotine.

For the uses, methods and compositions mentioned herein the dosage administered will, of course, vary with the compound employed, the mode of administration and the treatment desired.

The compounds of Formula I can be prepared by the synthetic routes illustrated in the following Scheme.

According to the Scheme, 3-methylenequinuclidine²² 1 is protected by formation of a boron complex on the tertiary nitrogen atom. Suitable boron complexes include: borane dimethyl sulfide, borane isoamylsulfide, borane tetrahydrofuran, borane pyridine, borane diphenylphosphine, 9-borabicyclo[3.3.1]nonane, boron tribromide, boron trichloride, boron trifluoride, (+)-isopinocamphenylborane TMEDA, (+)-B-chlorodiisopinocanphenylborane, (−)-B-chlorodiisopinocanphenylborane, (1S)-(+)-B-bromodiisopinocanphenylborane, (1R)-(−)-B-bromodiisopinocanphenylborane. The reaction is performed in an organic solvent. The preferred organic solvent is tetrahydrofuran. The reaction is carried out at a temperature of 0-100° C., and preferably at a temperature of 30° C.

In the subsequent step the 3-methylenequinuclidine boron complex is converted into the spirocyclic intermediate by means of a 1,3-dipolar cycloaddition reaction. The pericyclic reaction is performed between dipolarophile 2 and a 1,3-dipole in the presence of a base in an organic solvent solution or suspension. Suitable 1,3-dipoles include nitrile oxides and nitrile imines. Suitable organic bases include triethylamine, 1,8-diazabicyclo[5.4.0]undec-7-ene, 4-di(methylamino)pyridine, pyridine or potassium and sodium bases. Suitable inorganic bases include sodium and potassium bases. The reaction is performed in an organic solvent. The preferred solvent is ethyl acetate. The reaction is carried out at a temperature of 20-100° C.

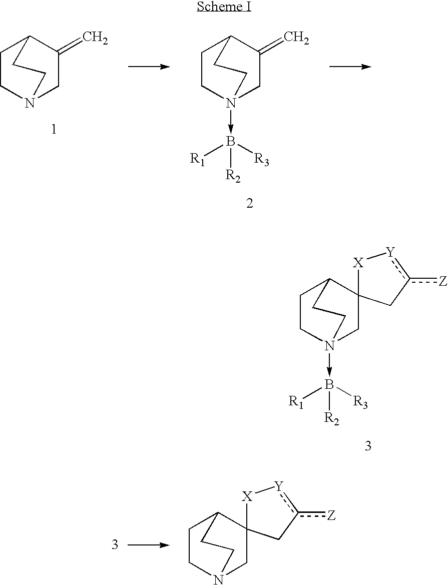

Acid addition salts of the compounds of formula I which may be mentioned include a) salts of mineral acids, such as the salts of halogenhydric, sulphuric, phosphoric acids, and salts formed with organic acids such as formic, acetic, maleic, benzoic, hydroxybenzoic, tartaric, malonic, fumaric, methanesulfonic, benzenesulfonic, toluenesulfonic acids and the like, and b) the methyl iodide salts (iodomethylates).

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, methanol, 2-propanol, tetrahydrofuran, or diethyl ether, or a mixture of solvents, which may be removed in vacuum or be freeze drying. The compounds of formula I exist in tautomeric or enantiomeric forms, all of which are included within the scope of the invention. The various optical isomers may be isolated by separation of a racemic mixture of the compounds using conventional techniques, e.g. fractional crystallization or chiral HPLC. Alternatively, the individual enantiomers may be made by reaction of the appropriate optically active starting materials under reaction conditions that will not cause racemization.

In the group of compounds of formula Ia, the levorotatory enantiomers are invariably endowed with higher affinity and efficacy than the corresponding dextrorotatory enantiomers.

### DESCRIPTION OF THE FIGURES

FIG. 1. Determination of the peak amplitudes of the currents induced by 500 μM concentrations of a compound selected among the derivatives of formula Ia in cells expressing the hα7 and hα4β2 receptor subtypes (values are normalized at the current induced by acetylcholine 200 μM).

FIG. 2. Effect of acute administration of a compound selected among the derivatives of formula Ia on scopolamine-induce amnesia in male Wistar rats in passive avoidance test. Amnesia was induced by scopolamine 0.125 mg/kg s.c. 30 min before the training session. The compound was given i.p. (5 mg/kg) (FIG. 2a) 20 minutes before the training session and was given p.o. (15 mg/kg) (FIG. 2b) 60 minutes before the training session.

### EXAMPLE 1

### (±)-3′-Ethyl-4′H-spiro[4-azabicyclo[2.2.2]octane-2,5′-isoxazole]

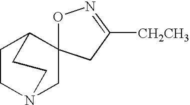

A. To a solution of borane-3-methylene-1-azabicyclo[2.2.2]octane complex²³ (1.0 g, 7.31 mmol) in dichloromethane (55 mL) were added a solution of propiohydroximoyl chloride²⁴ (1.179 g, 10.96 mmol) in dichloromethane (2 mL) and triethylamine (1.5 mL, 10.96 mmol). The reaction mixture was stirred at r. t. for 2 days with further addiction of amounts (3×3.0 g) of propiohydroximoyl chloride. After addition of water (50 mL), the reaction mixture was extracted with dichloromethane (3×50 mL). The pooled organic layers were dried over anhydrous sodium sulfate, filtered, and concentrated in vacuo. The residue was purified by silica gel column chromatography (eluant:petroleum ether/ethyl acetate 9:1) to yield the desired cycloadduct as a yellow oil (532 mg, 35% yield). R_{f}=0.32 (eluant: petroleum ether/ethyl acetate 2:3).

### (±)-3′-Ethyl-4-boranyl-4′H-spiro[4-azabicyclo[2.2.2]octane-2,5′-isoxazole]

¹H NMR (300 MHz, CDCl₃): δ 1.15 (t, 3H, J=7.4 Hz), 1.68 (m, 4H), 1.87 (m, 2H), 2.12 (q, 2H, J=7.4 Hz), 2.07 (m, 1H), 2.28 (m, 1H), 2.76 (d, 1H, J=17.2 Hz), 2.91-3.09 (m, 4H), 3.03 (d, 1H, J=17.2 Hz), 3.37 (d, 1H, J=14.6 Hz).

B. To an ice cooled and stirred solution of (±)-3′-ethyl-4-boranyl-4′H-spiro[4-azabicyclo[2.2.2]octane-2,5′-isoxazole] (333 mg, 1.60 mmol) in acetone (3 mL) a solution of trifluoroacetic acid (0.5 mL) in acetone (2.0 mL) was added dropwise and the disappearance of the starting material was monitored by TLC (eluant:dichloromethane/methanol 95:5). Toluene (5 mL) was added and the solvents and excess reagent were evaporated in vacuo. The residue was diluted with water (5 mL) and extracted with dichloromethane (3×5 mL). The residual aqueous phase, made basic by portionwise addition of K₂CO₃, was then extracted with dichloromethane (4×5 mL). After the usual work up, the crude base (221 mg, 71% yield) was obtained as a colorless oil. R_{f}=0.41 (eluant:dichloromethane/methanol 4:1).

### (±)-3′-Ethyl-4′H-spiro[4-azabicyclo[2.2.2]octane-2,5′-isoxazole]

¹H NMR (300 MHz, CDCl₃): δ 1.13 (t, 3H, J=7.4 Hz), 1.42 (m, 1H), 1.60 (m, 2H), 1.90 (m, 1H), 2.10 (q, 2H, J=7.4 Hz), 2.15 (m, 1H), 2.68 (d, 1H, J=17.2 Hz), 2.81 (m, 4H), 2.90 (d, 1H, J=14.6 Hz), 3.04 (d, 1H, J=17.2 Hz), 3.27 (d, 1H, J=14.6 Hz).

C. To a solution of (±)-3′-ethyl-4′H-spiro[4-azabicyclo[2.2.2]octane-2,5′-isoxazole] (214 mg, 1.10 mmol) in methanol (3 mL) was added a solution of fumaric acid (154 mg, 1.33 mmol) in methanol (2 mL). After stirring at r. t. for 16 h, the reaction mixture was concentrated under reduced pressure affording quantitatively the crude fumarate, which was crystallized from ethyl acetate/2-propanol (95:5).

### (±)-3′-Ethyl-4′H-spiro[4-azabicyclo[2.2.2]octane-2,5′-isoxazole]×¾Fumarate: colorless prisms, mp 168-170° C.

¹H NMR (300 MHz, D₂O): δ 1.18 (t, 3H, J=7.4 Hz), 1.70 (m, 2H), 1.86 (m, 1H), 1.90 (q, 2H, J=7.4 Hz), 2.07 (m, 2H), 2.92 (d, 1H, J=17.9 Hz), 3.07-3.25 (m, 5H), 3.38 (m, 2H), 6.49 (s, 1.5H).

¹³C NMR (75.4 MHz, D₂O): δ 10.5, 11.8, 18.2, 19.8, 29.8, 45.4, 46.0, 47.0, 59.2, 82.8, 135.0, 156.6, 170.2.

D. To a solution of (±)-3′-ethyl-4′H-spiro-[4-azabicyclo[2.2.2]octane-2,5′-isoxazole] (194 mg, 1.00 mmol) in methanol (3 mL) was added iodomethane (0.5 mL). After stirring at r. t. for 16 h, the solvent was evaporated to yield quantitatively the crude salt, which was crystallized from absolute ethanol and diethyl ether (3:7).

### (±)-3′-Ethyl-4′H-spiro[4-azabicyclo[2.2.2]octane-2,5′-isoxazole]Methyl iodide: colorless prisms, mp 210-212° C.

¹H NMR (300 MHz, D₂O): δ 1.16 (t, 3H, J=7.4 Hz), 1.80 (m, 2H), 1.93 (m, 1H), 1.94 (q, 2H, J=7.4 Hz), 2.15 (m, 2H), 2.83 (s, 3H), 2.95 (d, 1H, J=17.9 Hz), 3.13-3.42 (m, 5H), 3.52 (s, 2H).

¹³C NMR (75.4 MHz, CD₃OD): δ 11.0, 12.1, 19.7, 21.1, 29.6, 47.1, 51.4, 56.1, 56.7, 68.8, 83.4, 156.9.

### EXAMPLE 2

### (±)-3′-(Pyridin-3-yl)-4′H-spiro[4-azabicyclo[2.2.2]octane-2,5′-isoxazole]

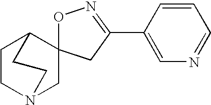

A. To a suspension of borane-3-methylene-1-azabicyclo[2.2.2]octane complex (1.0 g, 7.31 mmol) and 3-pyridinecarbohydroximoyl chloride²⁵ (1.144 g, 7.31 mmol) in toluene (80 mL) was added dropwise a solution of triethylamine (2.0 mL, 14.62 mmol) in toluene (10 mL). While heating at reflux under nitrogen for 3 days, further amounts of 3-pyridinecarbohydroximoyl chloride (2.288 g, 14.62 mmol) and triethylamine (4.0 mL, 29.24 mmol) were added portionwise to the reaction mixture. Water (30 mL) was added to the reaction mixture, the phases were separated and the aqueous layer was extracted with ethyl acetate (3×50 mL). After the usual work-up, the residue was purified by silica gel column chromatography (eluant:petroleum ether/ethyl acetate 4:1) to yield the desired cycloadduct (883 mg, 47% yield), which crystallized as colorless leaflets from ethyl acetate (mp 187-188.5° C.). R_{f}=0.28 (eluant:petroleum ether/ethyl acetate 3:2).

### (±)-3′-(Pyridin-3-yl)-4-boranyl-4′H-spiro[4-azabicyclo[2.2.2]octane-2,5′-isoxazole]

¹H NMR (300 MHz, CDCl₃): δ 1.82 (m, 2H), 2.02 (m, 1H), 2.30 (m, 1H), 2.42 (bs, 1H), 3.05-3.30 (m, 6H), 3.57 (m, 2H), 7.31 (m, 1H), 7.98 (d, 1H, J=7.9 Hz), 8.55 (bs, 1H), 8.72 (bs, 1H).

B. An ice cooled solution of (±)-3′-(pyridin-3-yl)-4-boranyl-4′H-spiro[4-azabicyclo[2.2.2]octane-2,5′-isoxazole] (530 mg, 2.06 mmol) was treated with trifluoroacetic acid following the above described procedure. The desired free base was obtained as a colorless viscous oil (341 mg, 68% yield). R_{f}=0.37 (eluant:dichloromethane/methanol 9:1).

### (±)-3′-(Pyridin-3-yl)-4′H-spiro[4-azabicyclo[2.2.2]octane-2,5′-isoxazole]

¹H NMR (300 MHz, CDCl₃): δ 1.44 (m, 1H), 1.65 (m, 2H), 2.07 (m, 1H), 2.10 (bs, 1H), 2.78-3.05 (m, 5H), 3.08 (d, 1H, J=16.8 Hz), 3.34 (d, 1H, J=15.4 Hz), 3.48 (d, 1H, J=16.8 Hz), 7.35 (dd, 1H, J=4.3 and 6.9 Hz), 8.04 (d, 1H, J=6.9 Hz), 8.63 (d, 1H, J=4.3 Hz), 8.75 (s, 1H).

C. The salts of (±)-3′-(pyridin-3-yl)-4′H-spiro[4-azabicyclo[2.2.2]octane-2,5′-isoxazole] with fumaric acid and methyl iodide were prepared by the above described protocols.

### (±)-3′-(Pyridin-3-yl)-4′H-spiro[4-azabicyclo[2.2.2]octane-2,5′-isoxazole]Fumarate: colorless prisms from 2-propanol, mp 197-198.5° C.

¹H NMR (300 MHz, D₂O): δ 1.72-2.00 (m, 3H), 2.08 (m, 2H), 3.08-3.37 (m, 4H), 3.42 (d, 1H, J=17.5 Hz), 3.49 (d, 1H, J=14.3 Hz), 3.55 (d, 1H, J=14.3 Hz), 3.66 (d, 1H, J=17.5 Hz), 6.58 (s, 2H), 7.56 (dd, 1H, J=4.4 and 7.0 Hz), 8.20 (d, 1H, J=7.0 Hz), 8.70 (d, 1H, J=4.4 Hz), 8.87 (s, 1H).

¹³C NMR (75.4 MHz, D₂O): δ 18.2, 19.4, 29.6, 44.4, 46.1, 46.7, 59.4, 84.7, 124.9, 126.8, 134.8, 137.8, 152.6, 154.2, 167.5, 171.4.

### (±)-3′-(Pyridin-3-yl)-4′H-spiro[4-azabicyclo[2.2.2]octane-2,5′-isoxazole]Methyl iodide: light yellow prisms from 2-propanol, mp 216-220° C., dec.

¹H NMR (300 MHz, D₂O): δ 1.75-2.08 (m, 3H), 2.15 (m, 2H), 2.87 (s, 3H), 3.10-3.65 (m, 7H), 3.69 (d, 1H, J=17.4 Hz), 7.59 (dd, 1H, J=4.4 and 7.1 Hz), 8.18 (d, 1H, J=7.1 Hz), 8.72 (d, 1H, J=4.4 Hz), 8.90 (s, 1H).

¹³C NMR (75.4 MHz, D₂O): δ 19.3, 20.9, 29.7, 44.5, 51.9, 56.4, 56.8, 67.9, 84.9, 125.0, 127.2, 137.9, 152.6, 154.3, 167.6.

### EXAMPLE 3

### (±)-3′-Benzyloxy-4′H-spiro[4-azabicyclo[2.2.2]octane-2,5′-isoxazole]

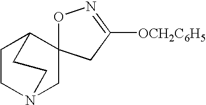

A. To a suspension of borane-3-methylene-1-azabicyclo[2.2.2]octane complex (4.50 g, 32.9 mmol) and potassium carbonate (22.7 g, 164 mmol) in ethyl acetate (90 mL) was added dibromoformaldoxime²⁶ (6.68 g, 32.9 mmol). The reaction mixture was stirred at r. t. for 5 days with further addition of amounts (5×2.0 g) of dibromoformaldoxime. After completion of the cycloaddition, Celite was added and the resulting slurry was filtered under vacuum and washed with ethyl acetate. The solvent was evaporated and the residue was purified by silica gel column chromatography (eluant:petroleum ether/ethyl acetate 1:1) to afford the wanted cycloadduct (4.05 g, 47% yield), which crystallized as colorless prisms from ethyl acetate (mp 127-128° C.). R_{f}=0.18 (eluant: petroleum ether/ethyl acetate 2:3).

### (±)-3′-Bromo-4-boranyl-4′H-spiro[4-azabicyclo[2.2.2]octane-2,5′-isoxazole]

¹H NMR (300 MHz, CDCl₃): δ 1.72 (m, 2H), 1.98 (m, 1H), 2.25 (bs, 1H), 2.33 (m, 1H), 2.98-3.18 (m, 6H), 3.30-3.44 (m, 2H).

B. To a solution of benzyl alcohol (0.30 mL, 2.90 mmol) in anhydrous tetrahydrofuran (3 mL) under nitrogen was added NaH (76 mg, 3.19 mmol). The reaction mixture was stirred at r. t. for 30 min, then cooled at 0° C. and a solution of (±)-3′-bromo-4-boranyl-4′H-spiro[4-azabicyclo[2.2.2]octane-2,5′-isoxazole (500 mg, 1.93 mmol) in anhydrous tetrahydrofuran (5 mL) was added dropwise. The mixture was stirred at r. t. for 3 h, water (1 mL) was added and the solvent was evaporated under vacuum. The residue was diluted with water (5 mL) and extracted with ethyl acetate (4×5 mL). The pooled organic layers ware dried over anhydrous sodium sulfate, filtered, and concentrated in vacuo. The crude reaction mixture was purified by silica gel column chromatography (eluant:petroleum ether/ethyl acetate 3:2) to afford the desired compound (428 mg, 77% yield), which crystallized as colorless prisms from ethyl acetate/n-hexane (mp 128-129° C.). R_{f}=0.44 (eluant:petroleum ether/ethyl acetate 2:3).

### (±)-3′-Benzyloxy-4-boranyl-4′H-spiro[4-azabicyclo[2.2.2]octane-2,5′-isoxazole]

¹H NMR (300 MHz, CDCl₃): δ 1.70 (m, 2H), 1.94 (m, 1H), 2.28 (bs, 1H), 2.35 (m, 1H), 2.90 (d, 1H, J=16.5 Hz), 2.98-3.13 (m, 6H), 3.41 (d, 1H, J=14.6 Hz), 5.13 (s, 2H), 7.37 (m, 5H).

C. An ice cooled solution of (±)-3′-benzyloxy-4-boranyl-4′H-spiro[4-azabicyclo[2.2.2]octane-2,5′-isoxazole] (350 mg, 1.22 mmol) was treated with trifluoroacetic acid following the above described procedure. The desired free base was obtained as a colorless viscous oil (264 mg, 79% yield). R_{f}=0.25 (eluant:dichloromethane/methanol 95:5).

### (±)-3′-Benzyloxy-4′H-spiro[4-azabicyclo[2.2.2]octane-2,5′-isoxazole]

¹H NMR (300 MHz, CDCl₃): δ 1.47 (m, 1H), 1.56 (m, 1H), 1.64 (m, 1H), 2.05 (bs, 1H), 2.14 (m, 1H), 2.68-2.90 (m, 4H), 2.79 (d, 1H, J=16.1 Hz), 2.94 (d, 1H, J=14.3 Hz), 3.07 (d, 1H, J=16.1 Hz), 3.24 (d, 1H, J=14.3 Hz), 5.12 (s, 2H), 7.37 (m, 5H).

D. The salts of (±)-3′-benzyloxy-4′H-spiro[4-azabicyclo[2.2.2]octane-2,5′-isoxazole] with fumaric acid and methyl iodide were prepared by the above described protocols.

### (±)-3′-Benzyloxy-4′H-spiro[4-azabicyclo[2.2.2]octane-2,5′-isoxazole]×¾Fumarate: colorless prisms from 2-propanol, mp 122-123° C.

¹H NMR (300 MHz, D₂O): δ 1.68 (m, 2H), 1.89 (m, 1H), 2.09 (m, 1H), 2.21 (bs, 1H), 2.97 (d, 1H, J=17.2 Hz), 3.03-3.24 (m, 4H), 3.17 (d, 1H, J=17.2 Hz), 3.40 (m, 2H), 4.97 (s, 2H), 6.47 (s, 1.5H), 7.25 (m, 5H).

¹³C NMR (75.4 MHz, D₂O): δ 17.6, 19.4, 28.9, 41.5, 45.9, 46.5, 58.5, 72.4, 84.2, 128.3, 128.9, 129.0, 134.7, 135.0, 167.8, 171.5.

### (±)-3′-Benzyloxy-4′H-spiro[4-azabicyclo[2.2.2]octane-2,5′-isoxazole] Methyl iodide: hygroscopic colorless prisms from absolute ethanol/diethyl ether, mp>50° C.

¹H NMR (300 MHz, D₂O): δ 1.75 (m, 2H), 1.94 (m, 1H), 2.13 (m, 1H), 2.65 (bs, 1H), 2.84 (s, 3H), 2.99 (d, 1H, J=17.1 Hz), 3.16 (d, 1H, J=17.1 Hz), 3.16-3.31 (m, 5H), 3.54 (m, 2H), 4.98 (s, 2H), 7.27 (m, 5H).

¹³C NMR (75.4 MHz, D₂O): δ 19.1, 20.8, 28.9, 41.5, 51.8, 56.3, 56.8, 68.0, 72.5, 84.7, 128.4, 128.9, 129.1, 135.0, 167.7.

The following compounds have been similarly prepared:

### (±)-3′-Bromo-4′H-spiro[4-azabicyclo[2.2.2]octane-2,5′-isoxazole]

Pale yellow oil, m/z 246 (MH⁺)

### (±)-3′-Chloro-4′H-spiro[4-azabicyclo[2.2.2]octane-2,5′-isoxazole]

Pale yellow oil, m/z 201 (MH⁺)

### (±)-4′H-Spiro[4-azabicyclo[2.2.2]octane-2,5′-isoxazole]

Colorless thick oil, m/z 167 (MH⁺)

### (±)-3′-Methyl-4H-spiro[4-azabicyclo[2.2.2]octane-2,5′-isoxazole]

Colorless oil, m/z 181 (MH⁺)

### (±)-3′-n-Propyl-4′H-spiro[4-azabicyclo[2.2.2]octane-2,5′-isoxazole]

Yellow oil, m/z 209 (MH⁺)

### (±)-3′-iso-propyl-4′H-spiro[4-azabicyclo[2.2.2]octane-2,5′-isoxazole]

Pale yellow oil, m/z 209 (MH⁺)

### (±)-3′-n-Butyl-4′H-spiro[4-azabicyclo[2.2.2]octane-2,5′-isoxazole]

Yellow oil, m/z 223 (MH⁺)

### (±)-3′-iso-Butyl-4′H-spiro[4-azabicyclo[2.2.2]octane-2,5′-isoxazole]

Pale yellow oil, m/z 223 (MH⁺)

### (±)-3′-tert-Butyl-4′H-spiro[4-azabicyclo[2.2.2]octane-2,5′-isoxazole]

Pale yellow oil, m/z 223 (MH⁺)

### (±)-3′-Methoxy-4′H-spiro[4-azabicyclo[2.2.2]octane-2,5′-isoxazole]

Colorless oil, m/z 197 (MH⁺)

### (±)-3′-Ethoxy-4′H-spiro[4-azabicyclo[2.2.2]octane-2,5′-isoxazole]

Colorless oil, m/z 211 (MH⁺)

### (±)-3′-n-Propoxy-4′H-spiro[4-azabicyclo[2.2.2]octane-2,5′-isoxazole]

Pale yellow oil, m/z 225 (MH⁺)

### (±)-3′-iso-Propoxy-4′H-spiro[4-azabicyclo[2.2.2]octane-2,5′-isoxazole]

Yellow oil, m/z 225 (MH⁺)

### (±)-3′-Butoxy-4′H-spiro[4-azabicyclo[2.2.2]octane-2,5′-isoxazole]

Colorless oil, m/z 239 (MH⁺)

### (±)-3′-iso-Butoxy-4′H-spiro[4-azabicyclo[2.2.2]octane-2,5′-isoxazole]

Colorless oil, m/z 239 (MH⁺)

### (±)-3′-tert-Butoxy-4′H-spiro[4-azabicyclo[2.2.2]octane-2,5′-isoxazole]

Colorless oil, m/z 239 (MH⁺)

### (±)-3′-Vinyl-4′H-spiro[4-azabicyclo[2.2.2]octane-2,5′-isoxazole]

Colorless oil, m/z 193 (MH⁺)

### (±)-3′-Allyl-4′H-spiro[4-azabicyclo[2.2.2]octane-2,5′-isoxazole]

Colorless oil, m/z 207 (MH⁺)

### (±)-3′-(Vinyloxy)-4′H-spiro[4-azabicyclo[2.2.2]octane-2,5′-isoxazole]

Colorless oil, m/z 209 (MH⁺)

### (±)-3′-(Allyloxy)-4′H-spiro[4-azabicyclo[2.2.2]octane-2,5′-isoxazole]

Colorless oil, m/z 223 (MH⁺)

### (±)-3′-Ethynyl-4′H-spiro[4-azabicyclo[2.2.2]octane-2,5′-isoxazole]

Yellow oil, m/z 191 (MH⁺)

### (±)-3′-(Prop-2-yn-1-yl)-4′H-spiro[4-azabicyclo[2.2.2]octane-2,5′-isoxazole]

Colorless oil, m/z 205 (MH⁺)

### (±)-3′-(But-3-yn-1-yl)-4′H-spiro[4-azabicyclo[2.2.2]octane-2,5′-isoxazole]

Yellow oil, m/z 219 (MH⁺)

### (±)-3′-(But-2-yn-1-yl-)4′H-spiro[4-azabicyclo[2.2.2]octane-2,5′-isoxazole]

Colorless oil, m/z 219 (MH⁺)

### (±)-3′-(Ethynyloxy)-4′H-spiro[4-azabicyclo[2.2.2]octane-2,5′-isoxazole]

Colorless oil, m/z 207 (MH⁺)

### (±)-3′-(Prop-2-yn-1-yloxy)-4′H-spiro[4-azabicyclo[2.2.2]octane-2,5′-isoxazole]

Pale yellow oil, m/z 221 (MH⁺)

### (±)-3′-(But-3-yn-1-yloxy)-4′H-spiro[4-azabicyclo[2.2.2]octane-2,5′-isoxazole]

Colorless oil, m/z 235 (MH⁺)

### (±)-3′-(But-2-yn-1-yloxy)-4′H-spiro[4-azabicyclo[2.2.2]octane-2,5′-isoxazole]

Colorless oil, m/z 235 (MH⁺)

### (±)-3′-Benzyl-4′H-spiro[4-azabicyclo[2.2.2]octane-2,5′-isoxazole]

Colorless oil, m/z 257 (MH⁺)

### (±)-3′-Phenyl-4′H-spiro[4-azabicyclo[2.2.2]octane-2,5′-isoxazole]

Pale yellow oil, m/z 243 (MH⁺)

### (±)-3′-Mesityl-4′H-spiro[4-azabicyclo[2.2.2]octane-2,5′-isoxazole]

Colorless viscous oil, m/z 285 (MH⁺)

### (±)-3′-(4-Chlorophenyl)-4′H-spiro[4-azabicyclo[2.2.2]octane-2,5′-isoxazole]

Colorless oil, m/z 277 (MH⁺)

### (±)-3′-(Pyridin-2-yl)-4′H-spiro[4-azabicyclo[2.2.2]octane-2,5′-isoxazole]

Colorless oil, m/z 244 (MH⁺)

### (±)-3′-(Pyridin-4-yl)-4′H-spiro[4-azabicyclo[2.2.2]octane-2,5′-isoxazole]

Colorless oil, m/z 244 (MH⁺)

### (±)-3′-(Pyrimidin-2-yl)-4′H-spiro[4-azabicyclo[2.2.2]octane-2,5′-isoxazole]

Colorless oil, m/z 245 (MH⁺)

### (±)-3′-(Pyrimidin-4-y2)-4′H-spiro[4-azabicyclo[2.2.2]octane-2,5′-isoxazole]

Yellow oil, m/z 245 (MH⁺)

### (±)-3′-(Pyrimidin-5-yl)-4′H-spiro[4-azabicyclo[2.2.2]octane-2,5′-isoxazole]

Yellow oil, m/z 245 (MH⁺)

### (±)-3′-(Pyrazin-2-yl)-4′H-spiro[4-azabicyclo[2.2.2]octane-2,5′-isoxazole]

Colorless oil, m/z 245 (MH⁺)

### (±)-3′-(2-Furyl)-4′H-spiro[4-azabicyclo[2.2.2]octane-2,5′-isoxazole]

Colorless oil, m/z 233 (MH⁺)

### (±)-3′-(3-Furyl)-4′H-spiro[4-azabicyclo[2.2.2]octane-2,5′-isoxazole]

Colorless oil, m/z 233 (MH⁺)

### (±)-3′-(2-Thienyl)-4′H-spiro[4-azabicyclo[2.2.2]octane-2,5′-isoxazole]

Yellow oil, m/z 249 (MH⁺)

### (±)-3′-(3-Thienyl)-4′H-spiro[4-azabicyclo[2.2.2]octane-2,5′-isoxazole]

Pale yellow oil, m/z 249 (MH⁺)

### (±)-3′-(1H-Pyrrol-2-yl)-4′H-spiro[4-azabicyclo[2.2.2]octane-2,5′-isoxazole]

Yellow oil, m/z 232 (MH⁺)

### (±)-3′-(1H-Pyrrol-3-yl)-4′H-spiro[4-azabicyclo[2.2.2]octane-2,5′-isoxazole]

Yellow oil, m/z 232 (MH⁺)

### (±)-3′-(Phenoxy)-4′H-spiro[4-azabicyclo[2.2.2]octane-2,5′-isoxazole]

Colorless oil, m/z 259 (MH⁺)

### (±)-3′-(Mesityloxy)-4′H-spiro[4-azabicyclo[2.2.2]octane-2,5′-isoxazole]

Yellow thick oil, m/z 301 (MH⁺)

### (±)-3′-(4-Chlorophenoxy)-4′H-spiro[4-azabicyclo[2.2.2]octane-2,5′-isoxazole]

Colorless oil, m/z 293 (MH⁺)

### (±)-3′-(Pyridin-2-yloxy)-4′H-spiro[4-azabicyclo[2.2.2]octane-2,5′-isoxazole]

Colorless oil, m/z 260 (MH⁺)

### (±)-3′-(Pyridin-3-yloxy)-4′H-spiro[4-azabicyclo[2.2.2]octane-2,5′-isoxazole]

Colorless oil, m/z 260 (MH⁺)

### (±)-3′-(Pyridin-4-yloxy)-4′H-spiro[4-azabicyclo[2.2.2]octane-2,5′-isoxazole]

Colorless oil, m/z 260 (MH⁺)

### (±)-3′-(Pyrimidin-2-yloxy)-4′H-spiro[4-azabicyclo[2.2.2]octane-2,5′-isoxazole]

Yellow oil, m/z 261 (MH⁺)

### (±)-3′-(Pyrimidin-4-yloxy)-4′H-spiro[4-azabicyclo[2.2.2]octane-2,5′-isoxazole]

Colorless oil, m/z 261 (MH⁺)

### (±)-3′-(Pyrimidin-5-yloxy)-4′H-spiro[4-azabicyclo[2.2.2]octane-2,5′-isoxazole]

Light orange oil, m/z 261 (MH⁺)

### (±)-3′-(Pyrazin-2-yloxy)-4′H-spiro[4-azabicyclo[2.2.2]octane-2,5′-isoxazole]

Colorless oil, m/z 261 (MH⁺)

### (±)-3′-(2-Furyloxy)-4′H-spiro[4-azabicyclo[2.2.2]octane-2,5′-isoxazole]

Pale yellow oil, m/z 249 (MH⁺)

### (±)-3′-(3-Furyloxy)-4′H-spiro[4-azabicyclo[2.2.2]octane-2,5′-isoxazole]

Colorless oil, m/z 249 (MH⁺)

### (±)-3′-(2-Thienyloxy)-4′H-spiro[4-azabicyclo[2.2.2]octane-2,5′-isoxazole]

Colorless oil, m/z 265 (MH⁺)

### (±)-3′-(3-Thienyloxy)-4′H-spiro[4-azabicyclo[2.2.2]octane-2,5′-isoxazole]

Colorless oil, m/z 265 (MH⁺)

### (±)-3′-(1H-Pyrrol-2-yloxy)-4′H-spiro[4-azabicyclo[2.2.2]octane-2,5′-isoxazole]

Yellow oil, m/z 248 (MH⁺)

### (±)-3′-(1H-Pyrrol-3-yloxy)-4′H-spiro[4-azabicyclo[2.2.2]octane-2,5′-isoxazole]

Dark yellow oil, m/z 248 (MH⁺)

### (±)-4′H-Spiro[4-azabicyclo[2.2.2]octane-2,5′-isoxazole]

Colorless oil, m/z 183 (MH⁺)

### (±)-4′H-Spiro[4-azabicyclo[2.2.2]octane-2,5′-isoxazole]-3′-ylmethanol

Colorless oil, m/z 197 (MH⁺)

### (±)-4′H-Spiro[4-azabicyclo[2.2.2]octane-2,5′-isoxazole]-3′-carbonitrile

Colorless oil, m/z 192 (MH⁺)

### (±)-Methyl-4′H-spiro[4-azabicyclo[2.2.2]octane-2,5′-isoxazole]-3′-carboxylate

Pale yellow oil, m/z 225 (MH⁺)

### (±)-Ethyl-4′H-spiro[4-azabicyclo[2.2.2]octane-2,5′-isoxazole]-3′-carboxylate

Colorless oil, m/z 239 (MH⁺).

### EXAMPLE 4

### (±)-5′-Bromo-2′-phenyl-2′,4′-dihydrospiro[4-azabicyclo[2.2.2]octane-2,3′-pyrazole]

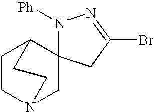

A. To a suspension of borane-3-methylene-1-azabicyclo[2.2.2]octane complex (1.0 g, 7.31 mmol) and potassium carbonate (5.050 g, 36.55 mmol) in ethyl acetate (50 mL) was added phenylcarbonohydrazonic dibromide²⁷ (2.032 g, 7.31 mmol). The reaction mixture was stirred at r. t. for 5 days with further addition of amounts (3×2.0 g) of hydrazonoyl dibromide. After completion of the cycloaddition, Celite was added and the resulting slurry was filtered under vacuum and washed with ethyl acetate. The solvent was evaporated and the residue was purified by silica gel column chromatography (eluant:petroleum ether/ethyl acetate 4:1) to afford the corresponding cycloadduct (952 mg, 39% yield), which crystallized as light yellow prisms from ethyl acetate/n-hexane (mp 148-149° C.). R_{f}=0.27 (eluant:petroleum ether/ethyl acetate 7:3).

### (±)-4-Boranyl-5′-Bromo-2′-phenyl-2′,4′-dihydrospiro[4-azabicyclo[2.2.2]octane-2,3′-pyrazole]

¹H NMR (300 MHz, CDCl₃): δ 1.73 (m, 2H), 1.90 (m, 1H), 2.24 (bs, 1H), 2.38 (m, 1H), 2.92-3.22 (m, 4H), 3.34 (d, 1H, J=17.4 Hz), 3.38-3.50 (m, 2H), 3.52 (d, 1H, J=17.4 Hz), 6.98 (m, 3H), 7.28 (m, 2H).

B. An ice cooled solution of (±)-4-boranyl-5′-bromo-2′-phenyl-2′,4′-dihydrospiro[4-azabicyclo[2.2.2]octane-2,3′-pyrazole] (502 mg, 1.5 mmol) was treated with trifluoroacetic acid following the above described procedure. The desired free base was obtained as a pale yellow viscous oil (245 mg, 51% yield).

### (±)-5′-Bromo-2′-phenyl-2′,4′-dihydrospiro[4-azabicyclo[2.2.2]octane-2,3′-pyrazole]

¹H NMR (300 MHz, CDCl₃): δ 1.52 (m, 1H), 1.66 (m, 1H), 1.70 (m, 1H), 2.11 (bs, 1H), 2.18 (m, 1H), 2.75-3.05 (m, 4H), 3.07-3.25 (m, 2H), 3.40 (d, 1H, J=17.3 Hz) 3.57 (d, 1H, J=17.3 Hz), 6.97 (t, 1H, J=7.5 Hz), 7.10 (2H, d, J=8.2 Hz), 7.28 (2H, dd, J=7.5 and 8.2 Hz).

C. The salts of (±)-5′-bromo-2′-phenyl-2′,4′-dihydrospiro[4-azabicyclo[2.2.2]octane-2,3′-pyrazole] with fumaric acid and methyl iodide were prepared by the above described protocols.

### (±)-5′-Bromo-2′-phenyl-2′,4′-dihydrospiro[4-azabicyclo[2.2.2]octane-2,3′-pyrazole]×¾Fumarate: colorless prisms from absolute ethanol/diethyl ether, mp 180-188° C., dec.

¹H NMR (300 MHz, D₂O): δ 1.70 (m, 2H), 1.92 (m, 1H), 2.12 (m, 1H), 2.27 (bs, 1H), 3.13-3.37 (m, 6H), 3.48 (d, 1H, J=17.4 Hz) 3.60 (d, 1H, J=17.4 Hz), 6.49 (s, 1.5H), 7.01 (t, 1H, J=7.4 Hz), 7.12 (2H, d, J=8.1 Hz), 7.32 (2H, dd, J=7.4 and 8.1 Hz).

¹³C NMR (75.4 MHz, D₂O): δ 18.0, 19.9, 29.2, 42.0, 46.5, 58.5, 63.4, 84.7, 129.2, 128.4, 130.2, 134.7, 135.1, 153.4, 166.2, 171.7.

### (±)-5′-Bromo-2′-phenyl-2′,4′-dihydrospiro[4-azabicyclo[2.2.2]octane-2,3′-pyrazole] Methyl iodide: light yellow prisms from 2-propanol, mp 203-207° C.

¹H NMR (300 MHz, D₂O): δ 1.76 (m, 2H), 2.00 (m, 1H), 2.15 (m, 1H), 2.58 (m, 1H), 2.88 (s, 3H), 3.20-3.42 (m, 6H), 3.50 (d, 1H, J=17.3 Hz), 3.64 (d, 1H, J=17.3 Hz), 7.04 (t, 1H, J=7.4 Hz), 7.10 (2H, d, J=8.2 Hz), 7.37 (2H, dd, J=7.4 and 8.2 Hz).

¹³C NMR (75.4 MHz, D₂O): δ 19.7, 21.6, 30.2, 42.7, 51.8, 56.9, 57.9, 60.8, 64.0, 84.1, 128.6, 130.8, 135.3, 153.6, 166.5.

The following compounds have been similarly prepared:

### (±)-5′-Bromo-2′,4′-dihydrospiro[4-azabicyclo[2.2.2]octane-2,3′-pyrazole]

Pale yellow oil, m/z 245 (MH⁺)
(±)-5′-Chloro-2′,4′-dihydrospiro[4-azabicyclo[2.2.2]octane-2,3′-pyrazole] Pale yellow oil, m/z 200 (MH⁺)
(±)-5′-Methoxy-2′,4′-dihydrospiro[4-azabicyclo[2.2.2]octane-2,3′-pyrazole] Pale yellow oil, m/z 196 (MH⁺)
(±)-5′-Methoxy-2′-phenyl-2′,4′-dihydrospiro[4-azabicyclo[2.2.2]octane-2,3′-pyrazole] Pale yellow oil, m/z 272 (MH⁺)
(±)-2′-Mesityl-5′-methoxy-2′,4′-dihydrospiro[4-azabicyclo[2.2.2]octane-2,3′-pyrazole] Pale yellow oil, m/z 314 (MH⁺)
(±)-2′-(4-Chlorophenyl)-5′-methoxy-2′,4′-dihydrospiro[4-azabicyclo[2.2.2]octane-2,3′-pyrazole] Yellow oil, m/z 306 (MH⁺)
(±)-2′-(4-Chlorophenyl)-5′-ethoxy-2 ′,4 ′-dihydrospiro[4-azabicyclo[2.2.2]octane-2,3′-pyrazole] Pale yellow oil, m/z 320 (MH⁺)
(±)-2′-(4-Chlorophenyl)-5′-(prop-2-yn-1-yloxy)-2′,4′-dihydrospiro[4-azabicyclo[2.2.2]octane-2,3′-pyrazole] Yellow oil, m/z 330 (MH⁺)
(±)-5′-Methyl-2′-phenyl-2′,4′-dihydrospiro[4-azabicyclo[2.2.2]octane-2,3′-pyrazole] Pale yellow oil, m/z 256 (MH⁺)
(±)-2′,5′-Diphenyl-2′,4′-dihydrospiro[4-azabicyclo[2.2.2]octane-2,3′-pyrazole] Yellow thick oil, m/z 318 (MH⁺)
(±)-5′-(2-Furyl)-2′-phenyl-2′,4′-dihydrospiro[4-azabicyclo[2.2.2]octane-2,3′-pyrazole] Colorless oil, m/z 308 (MH⁺)
(±)-2′-Phenyl-5′-(2-thienyl)-2′,4′-dihydrospiro[4-azabicyclo[2.2.2]octane-2,3′-pyrazole] Pale yellow oil, m/z 324 (MH⁺)
(±)-2′-Methyl-5′-(pyridin-2-yl)-2′,4′-dihydrospiro[4-azabicyclo[2.2.2]octane-2,3′-pyrazole] Pale yellow oil, m/z 257 (MH⁺)
(±)-2′-Methyl-5′-(pyridin-3-yl)-2′,4′-dihydrospiro[4-azabicyclo[2.2.2]octane-2,3′-pyrazole] Yellow oil, m/z 257 (MH⁺)
(±)-2′-Methyl-5′-(pyridin-4-yl)-2′,4′-dihydrospiro[4-azabicyclo[2.2.2]octane-2,3′-pyrazole] Pale yellow oil, m/z 257 (MH⁺)
(±)-5′-Methoxy-2′-methyl-2′,4′-dihydrospiro[4-azabicyclo[2.2.2]octane-2,3′-pyrazole] Pale yellow oil, m/z 210 (MH⁺).

### EXAMPLE 5

### (±)-2′-Methyl-3′H-spiro[4-azabicyclo[2.2.2]octane-2,5′-isoxazolidin]-3′-one

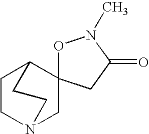

A. To a solution of (±)-3′-benzyloxy-4-boranyl-4′H-spiro[4-azabicyclo[2.2.2]octane-2,5′-isoxazole] (2.80 g, 9.79 mmol) in methanol (350 mL) and tetrahydrofuran (10 mL) was added Pd-C/10% (300 mg). The suspension was stirred under hydrogen for 2 days and a further amount (3×300 mg) of the catalyst was added. After completion of the reduction, the suspension was filtered and washed with methanol. The solvent was evaporated giving a colorless viscous oil (1.56 g, 81% yield). R_{f}=0.23 (eluant: dichloromethane/methanol 95:5).

### (±)-3′-Hydroxy-4-boranyl-4′H-spiro[4-azabicyclo[2.2.2]octane-2,5′-isoxazole]

¹H NMR (300 MHz, CDCl₃): δ 1.70 (m, 2H), 1.92 (m, 1H), 2.18 (m, 1H), 2.37 (bs, 1H), 2.70 (d, 1H, J=16.5 Hz), 2.81 (d, 1H, J=16.5 Hz), 2.93-3.24 (m, 5H), 3.32 (d, 1H, J=14.3 Hz).

B. To a solution of (±)-3′-hydroxy-4-boranyl-4′H-spiro[4-azabicyclo[2.2.2]octane-2,5′-isoxazole] (900 mg, 4.60 mmol) in acetone (30 mL) were added K₂CO₃ (1.90 9, 13.79 mmol) and iodomethane (2 mL). After stirring at r. t. for 16 h, the solvent was evaporated and the residue was diluted with water (8 mL) and extracted with dichloromethane (4×5 mL). The pooled organic layers ware dried over anhydrous sodium sulfate, filtered, and concentrated in vacuo. The crude product was purified by silica gel column chromatography (eluant: dichloromethane/methanol 95:5) giving a colorless oil (850 mg, 88% yield). R_{f}=0.51 (eluant: dichloromethane/methanol 95:5).

### (±)-2′-Methyl-4-boranyl-3′H-spiro[4-azabicyclo[2.2.2]octane-2,5′-isoxazolidin]-3′-one

¹H NMR (300 MHz, CDCl₃): δ 1.67 (m, 2H), 1.96 (m, 1H), 2.20 (m, 1H), 2.32 (bs, 1H), 2.66 (d, 1H, J=16.5 Hz), 2.82 (d, 1H, J=16.5 Hz), 2.96-3.14 (m, 5H), 3.16 (s, 3H), 3.29-3.40 (m, 1H).

C. An ice cooled solution of (±)-2′-methyl-4-boranyl-3′H-spiro[4-azabicyclo[2.2.2]octane-2,5′-isoxazolidin]-3′-one (500 mg, 2.38 mmol) was treated with trifluoroacetic acid following the procedure previously described. The desired free base was obtained as a colorless thick oil (271 mg, 58% yield).

### (±)-2′-Methyl-3′H-spiro[4-azabicyclo[2.2.2]octane-2,5′-isoxazolidin]-3′-one

¹H NMR (300 MHz, CDCl₃): δ 1.50 (m, 2H), 1.70 (m, 1H), 2.00 (m, 1H), 2.10 (bs, 1H), 2.16 (d, 1H, J=16.1 Hz), 2.72-3.09 (m, 7H), 3.14 (s, 3H).

D. The salts of (±)-2′-methyl-3′H-spiro[4-azabicyclo[2.2.2]octane-2,5′-isoxazolidin]-3′-one with fumaric acid and methyl iodide were prepared by the above described protocols.

### (±)-2′-Methyl-3′H-spiro[4-azabicyclo[2.2.2]octane-2,5′-isoxazolidin]-3′-one×½Fumarate: colorless prisms from ethyl acetate/methanol/n-hexane (96:2:2), mp 172-173° C.

¹H NMR (300 MHz, D₂O): δ 1.72 (m, 2H), 1.91 (m, 1H), 1.99 (m, 1H), 2.36 (bs, 1H), 2.82 (d, 1H, J=16.8 Hz), 2.82-3.31 (m, 5H), 3.03 (s, 3H), 3.38-3.59 (m, 2H), 6.52 (s, 1H).

¹³C NMR (75.4 MHz, D₂O): δ 17.4, 19.0, 28.5, 31.6, 41.6, 46.0, 46.4, 57.7, 80.7, 134.8, 168.4, 171.5.

### (±)-2′-Methyl-3′H-spiro[4-azabicyclo[2.2.2]octane-2,5′-isoxazolidin]-3′-one Methyl iodide: colorless prisms from 2-propanol, mp 182-183° C.

¹H NMR (300 MHz, D₂O): δ 1.82 (m, 2H), 1.99 (m, 1H), 2.22 (m, 1H), 2.42 (bs, 1H), 2.85 (m, 1H), 2.92 (s, 3H), 3.01 (m, 1H), 3.06 (s, 3H), 3.19-3.39 (m, 4H), 3.57-3.72 (m, 2H).

¹³C NMR (75.4 MHz, D₂O): δ 18.9, 20.4, 28.4, 31.6, 41.5, 51.8, 56.3, 56.7, 67.1, 81.1, 168.3.

The following compounds have been similarly prepared:
(±)-3′H-Spiro[4-azabicyclo[2.2.2]octane-2,5′-isoxazolidin]-3′-one Colorless oil, m/z 183 (MH⁺)
(±)-2′-Ethyl-3′H-spiro[4-azabicyclo[2.2.2]octane-2,5′-isoxazolidin]-3′-one Colorless oil, m/z 211 (MH⁺)
(±)-2′-n-Propyl-3′H-spiro[4-azabicyclo[2.2.2]octane-2,5′-isoxazolidin]-3′-one Colorless oil, m/z 225 (MH⁺)
(±)-2′-iso-Propyl-3′-spiro[4-azabicyclo[2.2.2]octane-2,5′-isoxazolidin]-3′-one Colorless oil, m/z 225 (MH⁺)
(±)-2′-n-Butyl-3′H-spiro[4-azabicyclo[2.2.2]octane-2,5′-isoxazolidin]-3′-one Pale yellow oil, m/z 239 (MH⁺)
(±)-2′-iso-Butyl-3′H-spiro[4-azabicyclo[2.2.2]octane-2,5′-isoxazolidin]-3′-one Yellow oil, m/z 239 (MH⁺)
(±)-2′-tert-Butyl-3′H-spiro[4-azabicyclo[2.2.2]octane-2,5′-isoxazolidin]-3′-one Colorless oil, m/z 239 (MH⁺)
(±)-2′-Allyl-3′H-spiro[4-azabicyclo[2.2.2]octane-2,5′-isoxazolidin]-3′-one Yellow oil, m/z 223 (MH⁺)
(±)-2′-(Prop-2-yn-1-yl)-3′H-spiro[4-azabicyclo[2.2.2]octane-2,5′-isoxazolidin]-3′-one Pale yellow oil, m/z 221 (MH⁺)
(±)-2′-(But-3-yn-1-yl)-3′H-spiro[4-azabicyclo[2.2.2]octane-2,5′-isoxazolidin]-3′-one Light orange oil, m/z 235 (MH⁺)
(±)-2′-Phenyl-3′H-spiro[4-azabicyclo[2.2.2]octane-2,5′-isoxazolidin]-3′-one Colorless viscous oil, m/z 259 (MH⁺)
(±)-2′-Mesityl-3′H-spiro[4-azabicyclo[2.2.2]octane-2,5′-isoxazolidin]-3′-one Pale yellow thick oil, m/z 301 (MH⁺)
(±)-2′-(4-Chlorophenyl)-3′H-spiro[4-azabicyclo[2.2.2]octane-2,5′-isoxazolidin]-3′-one Pale yellow oil, m/z 293 (MH⁺)
(±)-2′-Benzyl-3′H-spiro[4-azabicyclo[2.2.2]octane-2,5′-isoxazolidin]-3′-one Colorless oil, m/z 273 (MH⁺)
(±)-2′-(Pyridin-2-yl)-3′H-spiro[4-azabicyclo[2.2.2]octane-2,5′-isoxazolidin]-3′-one; yellow oil, m/z 260 (MH⁺)
(±)-2′-(Pyridin-3-yl)-3′H-spiro[4-azabicyclo[2.2.2]octane-2,5′-isoxazolidin]-3′-one Pale yellow oil, m/z 260 (MH⁺)
(±)-2′-(Pyridin-4-yl)-3′H-spiro[4-azabicyclo[2.2.2]octane-2,5′-isoxazolidin]-3′-one Yellow oil, m/z 260 (MH⁺)
(±)-2′-(2-Thienyl)-3′H-spiro[4-azabicyclo[2.2.2]octane-2,5′-isoxazolidin]-3′-one Pale yellow oil, m/z 265 (MH⁺)
(±)-2′-(3-Thienyl)-3′H-spiro[4-azabicyclo[2.2.2]octane-2,5′-isoxazolidin]-3′-one Yellow oil, m/z 265 (MH⁺).

Biological Activity

The compounds according to the invention, prepared by means of the synthetic approach illustrated above, were at first subjected to receptor affinity tests, by performing competitive binding tests at the α4β2 and α7 nicotinic receptors subtypes. The compounds of the invention show binding affinities (Kᵢ) lower than 1000 nM at α7 nicotinic receptors subtypes. The most interesting compounds, i.e. those characterized by significant affinity for the α7 nicotinic receptor subtype (Kᵢ=4-80 nM), as well as marked functional selectivity, were further assayed in electrophysiological experiments to evaluate their agonist/partial agonist profile and in a pharmacological in vivo test to investigate their effect on the cognitive behaviour.

Receptor Binding Assay

Membranes binding of [³H]-Epibatidine and [¹²⁵I]-α-Bungarotoxin (α-BTX): The cortex tissues were dissected, immediately frozen on dry ice and stored at 80° C. for later use. In each experiment, the cortex tissues from two rats were homogenized in 10 mL of a buffer solution (50 mM Na₃PO₄, 1 M NaCl, 2 mM EDTA, 2 mM EGTA and 2 mM PMSF, pH 7.4) using a potter homogenizer; the homogenates were then diluted and centrifuged at 60.000 g for 1.5 h. The total membrane homogenization, dilution and centrifugation procedures were performed twice, then the pellets were collected, rapidly rinsed with a buffer solution (50 mM Tris-HCl, 120 mM NaCl, 5 mM KCl, 1 mM MgCl₂, 2.5 mM CaCl₂ and 2 mM PMSF, pH 7), and resuspended in the same buffer containing a mixture of 20 μg/mL of each of the following protease inhibitors: leupeptin, bestatin, pepstatin A, and aprotinin.

[³H]-Epibatidine binding: (±)-[³H]-epibatidine with a specific activity of 56-60 Ci/mmol was purchased from Perkin Elmer (Boston Mass.); the non radioactive α-BTX, nicotine and epibatidine were purchased from Sigma. It has been previously reported that [³H]-epibatidine also binds to α-BTX binding receptors with nM affinity.²⁸ In order to prevent the binding of [³H]-epibatidine to the α-BTX binding receptors, the membrane homogenates were preincubated with 2 μM α-BTX and then with [³H]-epibatidine. The saturation experiments were performed by incubating aliquots of cortex membrane homogenates with 0.01-2.5 nM concentrations of (±)-[³H]-epibatidine overnight at 4° C. Non-specific binding was determined in parallel by means of incubation in the presence of 100 nM unlabeled epibatidine. At the end of the incubation, the samples were filtered on a GFC filter soaked in 0.5% polyethylenimine and washed with 15 mL of a buffer solution (10 mM Na₃PO₄, 50 mM NaCl, pH 7.4) and the filters were counted in a β counter.

[¹²⁵I]-α-BTX binding: The saturation binding experiments were performed using aliquots of cortex membrane homogenates incubated overnight with 0.1-10 nM concentrations of [¹²⁵I]-α-BTX (specific activity 200-213 Ci/mmol, Amersham) at r. t. Non-specific binding was determined in parallel by means of incubation in the presence of 1 μM unlabeled α-BTX. After incubation, the samples were filtered as described above and the bound radioactivity was directly counted in a γ counter.

nACh Receptor affinity of the investigated compounds: The inhibition of radioligand binding by epibatidine, nicotine and the test compounds was measured by preincubating cortex homogenates with increasing doses (10 pM-10 mM) of the reference nicotinic agonists, epibatidine or nicotine, and the drug to be tested for 30 min at r. t., followed by overnight incubation with a final concentration of 0.075 nM [³H]-epibatidine or 1 nM [¹²⁵I]-α-BTX at the same temperatures as those used for the saturation experiments. These ligand concentrations were used for the competition binding experiments because they are within the range of the K_{d} values of the ligands for the two different classes of nAChRs. For each compound, the experimental data obtained from the four saturation and four competition binding experiments were analyzed by means of a non-linear least square procedure, using the LIGAND program as described by Munson and Rodbard.²⁹ The binding parameters were calculated by simultaneously fitting four independent saturation experiments and the Kᵢ values were determined by fitting the data of four independent competition experiments. The errors in the K_{d} and Kᵢ values of the simultaneous fits were calculated using the LIGAND software, and were expressed as percentage coefficients of variation (% CV). When final compound concentrations up to 200 μM did not inhibit radioligand binding, the Kᵢ value was defined as being >100 μM based on the Cheng and Prusoff's equation.³⁰

Transfection of Human Subtypes

α7 Subtype: The human α7 (hα7) receptors were stably expressed in transfected GH4C1 and grown in the presence of geneticin (G418 sulfate, 500 μg/mL) as previously described.³¹

α4β2 Subtype: Transient transfections of the human nAChR α4 and β2 subunits were carried out in the retroviral packaging cell line HEK 293, as previously described³² using an optimized calcium phosphate procedure. The cells were grown in Dulbecco's modified Eagle's medium (DMEM, Gibco) supplemented with 10% fetal calf serum (Hyclone, USA). The subunit cDNAs were added in equivalent amounts (1 μg each per 100 mm dish). Between 8 and 12 h after transfection, the cells were washed twice and fed again with DMEM-containing 10% fetal calf serum.

Electrophysiological recordings: The standard protocol used for the experiments has been previously described.³⁰ Cells held at 50 mV were continuously superfused with control, acetylcholine or compound to be tested via independent tubes and connected to a fast exchanger system as previously reported.³³

Effects of compounds on acetylcholine-induced current amplitude: Whole cell patched-clamped hα7- and hα4β2-expressing cells exhibited inward currents that quickly decayed when challenged with acetylcholine. Specifically acetylcholine (200 μM) elicited a mean of 200±50 pA (n=8) for the hα7 subtype and 185±40 pA (n=8) for the hα4β2 subtype. The hα7 current was blocked by 50 nM methyllycaconitine and the hα4β2 current by 1 mM dihydro-β-erythroidine.

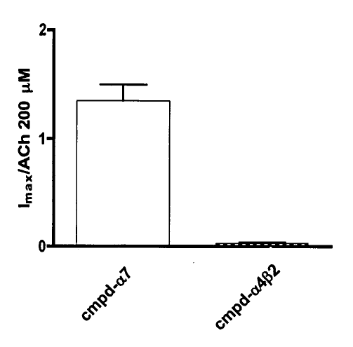

When a compound under study (compound in FIG. 1 is a representative example among the derivatives of formula Ia) was applied, it induced in a dose dependent manner an inward current in cells expressing the hα7 receptors. The peak amplitudes of this current was measured, normalized to the current induced by 200 μM acetylcholine, and the results are shown in FIG. 1. When tested on the cells expressing the hα4β2 subtype, the compound under investigation was much less potent in inducing inward currents; in fact, at a maximal concentration of 500 μM, it only elicited a 2% of the current induced by 200 μM acetylcholine. Therefore, the derivative had negligible effects on the α4β2 receptor subtype.

Cognitive Behaviour

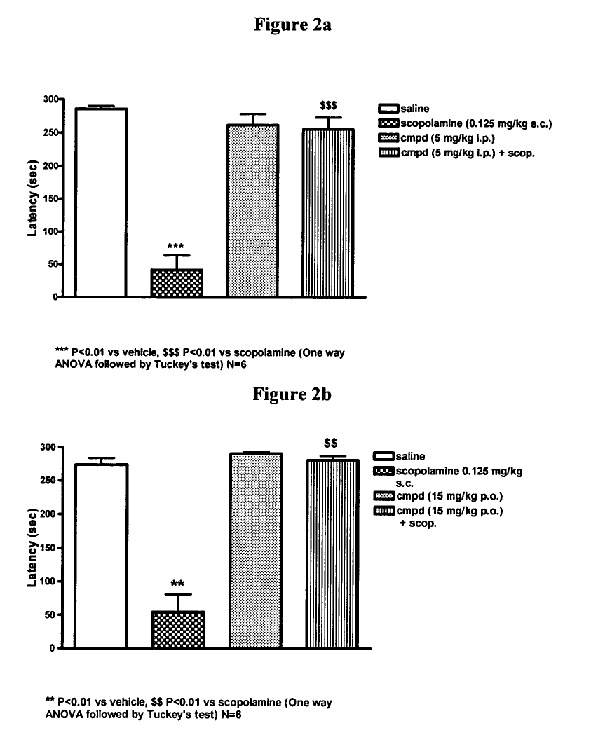

Cognitive behaviour was studied for selected compounds from example using the passive avoidance (PA) task in order to test the capability to reverse scopolamine-induced amnesia in rats. The compounds showed mild to good cognitive improvement of long term reference memory by inducing significant reversion of the scopolamine-induced amnesia (a representative result is shown in FIG. 2 for a compound selected from the derivatives of formula Ia).

Animals: Male Wistar rats weighing 180-220 g were housed in standard laboratory conditions for 7 days after their arrival. The day before the test they were divided in single cages.

Apparatus and procedures: The apparatus (Ugo Basile, Varese, Italy) consisted of a box divided by a guillotine door into two compartments of the same size (24×21×27) in which the floor had a grid of stainless rod. One compartment was lit with a 10 Watt electric bulb and had white walls, the other compartment was dark. The step-through type passive avoidance task was used as described by Ader³⁴ with slight modifications. On the first day (training session) rats were placed in the light compartment and allowed to enter the dark; once they did so, the door was automatically closed and an unavoidable scrambled footshock (0.8 mA) was delivered for 5 s. 24 h later rats were once again placed in the light compartment and the latency to re-enter the dark compartment was recorded up to a max of 300 s.

Treatment: The animals received a s.c. injection (1 ml/kg) of saline or scopolamine (0.125 mg/kg) 30 min before the training session. Saline or the compound (5 mg/kg) were given i.p. (5 ml/kg) 20 minutes before the training session. Saline or the compound (15 mg/kg) were given p.o. 60 minutes before the training session.

### REFERENCES

1. Jensen, A. A. et al., J. Med. Chem. 2005, 48, 4705-4745.
2. Séguéla, P. et al., J. Neurosci. 1993, 13, 596-604.
3. Weiland, S. et al., Behav. Brain Res. 2000, 113, 43-56.
4. Dajas-Bailador, F. et al., Trends Pharmacol. Sci. 2004, 25, 317-324.
5. Levin, E. D. et al., Psychopharmacology 1998, 138, 217-230.
6. Raux, G. et al., Mol. Psychiatry 2002, 7, 1006-1011.
7. Luntz-Leybman, V. et al., Brain Res. 1992, 587, 130-136.
8. Guan, Z. et al., Neuroreport 1999, 10, 1779-1782.
9. Hashimoto, K. et al., Curr. Med. Chem. 2005, 5, 171-184.
10. Leonard, S. et al., Pharmacol. Biochem. Behav. 2000, 70, 561-570.
11. Court, J. et al., J. Neurochem. 1999, 73, 1590-1597.
12. Wishka, D. G. et al., WO 0100857 (2002).
13. Toma, L. et al., Expert Opin. Ther. Patents 2004, 14, 1029-1040.
14. Tatsumi, R. et al. J. Med. Chem. 2005, 48, 905-908.
15. Gaviraghi, G. et al., WO 008133 (2006).
16. Biton, B. et al., Neuropsychopharmacology 2007, 32, 1-16.
17. Pichat, P. et al., Neuropsychopharmacology 2007, 32, 17-34.
18. Wang, H. et al., Nature 2003, 421, 384-387.
19. Ulloa, L. Nature Rev. Drug Discov. 2005, 4, 673-684.
20. Groppi Jr., V. E. et al., WO 04/064836 (2004).
21. Wang, H. et al., Nat. Med. 2004, 10, 1216-1221.
22. Lee, C. Y. L. et al., Arkivoc 2006 (iii), 175-183.
23. Stotter, P. L. et al., Heterocycles 1987, 25, 251-258.
24. Kumaran, G., Tetrahedron Lett. 1996, 37, 6407-6408.
25. Dallanoce, C. et al., Eur. J. Org. Chem. 2006, 3746-3754, and references cited therein.
26. Conti, P. et al., II Farmaco 2002, 57, 889-895, and references cited therein.
27. Foti, F. et al., Tetrahedron Lett. 1999, 40, 2605-2606.
28. Gerzanich, V. et al., Mol. Pharmacol. 1995, 48, 774-782.
29. Munson, P. J. et al., Anal. Biochem. 1980, 107, 220-239.
30. Cheng, Y.-C. et al., Biochem. Pharmacol. 1973, 22, 3099-3108.
31. Ragozzino, D. et al., Eur. J. Neurosci. 1997, 9, 480-488.
32. Lax, P. et al., Cell. Calcium 2002, 32, 53-58.
33. Fucile, S. et al., Cell. Calcium 2003, 34, 205-209.
34. Ader, R. et al., Psychonomic Science 1972, 26, 125-132.

1. Compounds of formula I: 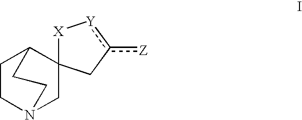
or pharmaceutically acceptable salts thereof, wherein:
a) when X is oxygen and Y is nitrogen, then, in the Y═C-Z moiety,
Z is selected from halogen; hydrogen; linear, branched or cyclic (C₁-C₆) alkyl, haloalkyl, alkoxy or acyl; (C₂-C₆) alkenyl, alkenyloxy; (C₂-C₆) alkynyl, alkynyloxy; benzyl, benzyloxy, (Ar) aryl, aryloxy; hydroxy; hydroxymethyl; cyano; nitro; amino; mono- or di-(C₁-C₆) alkylamino, aminomethyl, alkylaminomethyl, acylamino, alkylaminocarbonyl groups; linear, branched or cyclic (C₁-C₆) alkoxy-, (C₂-C₆) alkenyloxy-, (C₂-C₆) alkynyloxy- or (Ar) aryloxy-carbonyl groups;
Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar mojety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl
with the proviso that Z is not methyl, tert-butyl, phenyl or 2,4,6-trimethyl-phenyl;
b) when X is a group NR,
R is selected from hydrogen, linear, branched or cyclic (C₁-C₆) alkyl, benzyl, (Ar) aryl;
Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar mojety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl
and Y is nitrogen, then, in the Y═C-Z moiety,
Z is selected from halogen; hydrogen; linear, branched or cyclic (C₁-C₆) alkyl, haloalkyl, alkoxy or acyl; (C₂-C₆) alkenyl, alkenyloxy; (C₂-C₆) alkynyl, alkynyloxy; benzyl, benzyloxy, (Ar) aryl, aryloxy; hydroxy; hydroxymethyl; cyano; nitro; mono- or di-(C₁-C₆) alkylamino, aminomethyl, alkylaminomethyl, acylamino, alkylaminocarbonyl groups; linear, branched or cyclic (C₁-C₆) alkoxy-, (C₂-C₆) alkenyloxy-, (C₂-C₆) alkynyloxy- or aryloxy-carbonyl groups;
Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar mojety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl
c) when X is oxygen and Y is a group NR
R is selected from hydrogen; linear, branched or cyclic (C₁-C₆) alkyl, (C₂-C₆) alkenyl, (C₂-C₆) alkynyl; benzyl; (Ar) aryl
Ar is selected from unsubstitued phenyl; 2-pyridyl; 3-pyridyl or 4-pyridyl; 2-pyrimidyl, 4-pyrimidyl or 5-pyrimidyl; 2-pyrazinyl or 3-pyrazinyl; 2-furyl or 3-furyl; 2-thiophenyl or 3-thiophenyl; 1-pyrrolyl, 2-pyrrolyl or 3-pyrrolyl; 2-quinazolyl, 4-quinazolyl or 5-quinazolyl; 2-oxazolyl, 4-oxazolyl or 5-oxazolyl; 2-imidazolyl, 4-imidazolyl or 5-imidazolyl; 1-naphthyl or 2-naphthyl; 2-quinolyl, 3-quinolyl, 4-quinolyl, 5-quinolyl, 6-quinolyl, 7-quinolyl or 8-quinolyl; 1-isoquinolyl, 3-isoquinolyl, 4-isoquinolyl, 5-isoquinolyl, 6-isoquinolyl, 7-isoquinolyl or 8-isoquinolyl; 2-benzofuranyl, 3-benzofuranyl, 4-benzofuranyl, 5-benzofuranyl, 6-benzofuranyl or 7-benzofuranyl, 2-benzo[b]thiophenyl, 3-benzo[b]thiophenyl, 4-benzo[b]thiophenyl, 5-benzo[b]thiophenyl, 6-benzo[b]thiophenyl or 7-benzo[b]thiophenyl; 2-indolyl, 3-indolyl, 4-indolyl, 5-indolyl, 6-indolyl or 7-indolyl; 2-benzoxazolyl, 3-benzoxazolyl, 4-benzoxazolyl, 5-benzoxazolyl, 6-benzoxazolyl or 7-benzoxazolyl; 2-benzothiazolyl, 4-benzothiazolyl, 5-benzothiazolyl, 6-benzothiazolyl or 7-benzothiazolyl; or is selected from any foregoing Ar mojety substituted with one to three substituents selected from linear, branched or cyclic (C₁-C₆) alkyl, alkoxy; (C₂-C₆) alkenyl, alkenyloxy; (C₂-C₆) alkynyl, alkynyloxy; halogen; cyano; nitro; amino, mono- or di-(C₁-C₆) alkylamino, aminomethyl
then, in the Y—C=Z moiety, Z is oxygen.

2. The compounds according to claim 1, the compounds having formula Ia 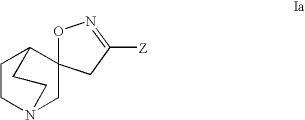
wherein Z is selected from Br, Cl; H; C₂H₅, n-C₃H₇, CH(CH₃)₂, n-C₄H₉, CH₂CH(CH₃)₂, OCH₃, OC₂H₅, O-n-C₃H₇, OCH(CH₃)₂, O-n-C₄H₉, OCH₂CH(CH₃)₂, OC(CH₃)₃; CH═CH₂, CH₂—CH═CH₂, OCH═CH₂, OCH₂—CH═CH₂; C≡CH, CH₂—C≡CH, C₂H₄—C≡CH, CH₂—C≡C—CH₃, OC≡CH, OCH₂—C≡CH, OC₂H₄—C≡CH, OCH₂—C≡C—CH₃; CH₂—C₆H₅, OCH₂—C₆H₅; 4-chloro-phenyl, 2-pyridyl, 3-pyridyl, 4-pyridyl; 2-pyrimidyl, 4-pyrimidyl, 5-pyrimidyl; 2-pyrazinyl; 2-furyl, 3-furyl; 2-thiophenyl, 3-thiophenyl; 2-pyrrolyl, 3-pyrrolyl, OC₆H₅, O-4-chloro-phenyl, O-2,4,6-trimethyl-phenyl, O-2-pyridyl, O-3-pyridyl, O-4-pyridyl; O-2-pyrimidyl, O-4-pyrimidyl, O-5-pyrimidyl; O-2-pyrazinyl, O-3-pyrazinyl; O-2-furyl, O-3-furyl; O-2-thiophenyl, O-3-thiophenyl; O-2-pyrrolyl, O-3-pyrrolyl; OH, CH₂OH; CH₂NH₂; CN; COOCH₃, COOC₂H₅, COOPh, COOCH₂Ph.

3. The compounds according to claim 1, the compounds having formula Ib 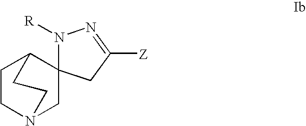
wherein:
R is selected from H, CH₃, C₂H₅, n-C₃H₇, CH(CH₃)₂, CH₂—C₆H₅, phenyl, 2,4,6-trimethyl-phenyl, 4-chloro-phenyl; 2-pyridyl, 3-pyridyl, 4-pyridyl, 2-thiophenyl, 3-thiophenyl,
and Z is selected from Br, Cl; H; CH₃, C₂H₅, n-C₃H₇, CH(CH₃)₂, n-C₄H₉, CH₂CH(CH₃)₂, C(CH₃)₃, OCH₃, OC₂H₅, O-n-C₃H₇, OCH(CH₃)₂, O-n-C₄H₉, OCH₂CH(CH₃)₂, OC(CH₃)₃; CH═CH₂, CH₂—CH═CH₂, OCH═CH₂, OCH₂—CH═CH₂; C≡CH, CH₂—C≡CH, C₂H₄—C≡CH, CH₂—C≡C—CH₃, OC≡CH, OCH₂—C≡CH, OC₂H₄—C≡CH, OCH₂—C≡C—CH₃; CH₂—C₆H₅, OCH₂—C₆H₅; phenyl, 2,4,6-trimethyl-phenyl, 4-chloro-phenyl, 2-pyridyl, 3-pyridyl, 4-pyridyl; 2-pyrimidyl, 4-pyrimidyl, 5-pyrimidyl; 2-pyrazinyl, 2-furyl, 3-furyl; 2-thiophenyl, 3-thiophenyl; 2-pyrrolyl, 3-pyrrolyl, OC₆H₅, O-4-chloro-phenyl, O-2,4,6-trimethyl-phenyl, O-2-pyridyl, O-3-pyridyl, O-4-pyridyl; O-2-pyrimidyl, O-4-pyrimidyl, O-5-pyrimidyl; O-2-pyrazinyl, O-3-pyrazinyl; O-2-furyl, O-3-furyl; O-2-thiophenyl, O-3-thiophenyl; O-2-pyrrolyl, O-3-pyrrolyl; OH, CH₂OH; CH₂NH₂; CN; COOCH₃, COOC₂H₅, COOPh, COOCH₂Ph.

4. The compounds according to claim 1, the compounds having formula Ic 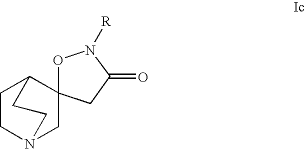
wherein R is selected from H, CH₃, C₂H₅, n-C₃H₇, CH(CH₃)₂, n-C₄H₉, CH₂CH(CH₃)₂, C(CH₃)₃; CH₂—CH═CH₂; CH₂—C≡CH, C₂H₄—C≡CH; phenyl, 2,4,6-trimethyl-phenyl, 4-chloro-phenyl, CH₂—C₆H₅; 2-pyridyl, 3-pyridyl, 4-pyridyl, 2-thiophenyl, 3-thiophenyl.

5. A medicament for diseases or conditions treatable through action of nicotinic acetylcholine receptors, the medicament comprising the compound of formula I as defined in claim 1 or a pharmaceutically acceptable salt thereof.

6. The medicament according to claim 5, wherein the nicotinic acetylcholine receptors are α 7 nicotinic acetylcholine receptors.

7. The medicament according to claim 6, wherein the diseases or conditions are neurological disorders, psychotic disorders or intellectual impairment disorders.

8. The medicament according to claim 7, wherein the diseases or conditions are Alzheimer's disease, learning deficit, cognition deficit, attention deficit, memory loss, Attention Deficit Hyperactivity Disorder, Parkinson's disease, Huntington's disease, Tourette's syndrome or neurodegenerative disorders in which there is loss of cholinergic synapses, anxiety, schizophrenia or mania or manic depression.

9. The medicament according to claim 5, wherein the diseases are inflammatory diseases or conditions.

10. The medicament according to claim 5, wherein the diseases or conditions are jetlag, cessation of smoking, nicotine addiction, craving, pain or ulcerative colitis.

11. A pharmaceutical composition comprising the compound of claim 1 and a pharmaceutically-acceptable diluent or carrier.

12. A method of treatment or prophylaxis in a human of diseases or conditions in which activation of a nicotinic acetylcholine receptors is beneficial for the human, the method comprising
administering to the human a therapeutically effective amount of the compound of claim 1.

13. α 7 nAChRs agonists comprising compounds of formula Ia as defined in claim 2, wherein Z is methyl, tert-butyl, phenyl or 2,4,6,-trimethyl-phenyl.

14. The method of claim 12, wherein the nicotinic acetylcholine receptors are α 7 nicotinic acetylcholine receptors.

15. The method according to claim 14, wherein the diseases or conditions are neurological disorders, psychotic disorders or intellectual impairment disorders.

16. The method according to claim 14, wherein the diseases or conditions are Alzheimer's disease, learning deficit, cognition deficit, attention deficit, memory loss, Attention Deficit Hyperactivity Disorder, Parkinson's disease, Huntington's disease, Tourette's syndrome or neurodegenerative disorders in which there is loss of cholinergic synapses, anxiety, schizophrenia or mania or manic depression.

17. The method according to claim 14, wherein the diseases or conditions are inflammatory diseases or conditions.

18. The method according to claim 14, wherein the diseases or conditions are jetlag, cessation of smoking, nicotine addiction, craving, pain or ulcerative colitis.

19. The pharmaceutical composition according to claim 11, wherein the compound has formula Ia 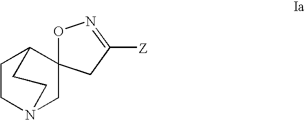
wherein Z is selected from Br, Cl; H; C₂H₅, n-C₃H₇, CH(CH₃)₂, n-C₄H₉, CH₂CH(CH₃)₂, OCH₃, OC₂H₅, O-n-C₃H₇, OCH(CH₃)₂, O-n-C₄H₉, OCH₂CH(CH₃)₂, OC(CH₃)₃; CH═CH₂, CH₂—CH═CH₂, OCH═CH₂, OCH₂—CH═CH₂; C≡CH, CH₂—C≡CH, C₂H₄—C≡CH, CH₂—C≡C—CH₃, OC≡CH, OCH₂—C≡CH, OC₂H₄—C≡CH, OCH₂—C≡C—CH₃; CH₂—C₆H₅, OCH₂—C₆H₅; 4-chloro-phenyl, 2-pyridyl, 3-pyridyl, 4-pyridyl; 2-pyrimidyl, 4-pyrimidyl, 5-pyrimidyl; 2-pyrazinyl; 2-furyl, 3-furyl; 2-thiophenyl, 3-thiophenyl; 2-pyrrolyl, 3-pyrrolyl, OC₆H₅, O-4-chloro-phenyl, O-2,4,6-trimethyl-phenyl, O-2-pyridyl, O-3-pyridyl, O-4-pyridyl; O-2-pyrimidyl, O-4-pyrimidyl, O-5-pyrimidyl; O-2-pyrazinyl, O-3-pyrazinyl; O-2-furyl, O-3-furyl; O-2-thiophenyl, O-3-thiophenyl; O-2-pyrrolyl, O-3-pyrrolyl; OH, CH₂OH; CH₂NH₂; CN; COOCH₃, COOC₂H₅, COOPh, COOCH₂Ph.

20. The pharmaceutical composition according to claim 11, wherein the compound has formula Ib 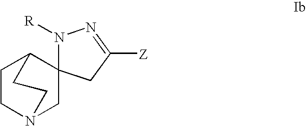
wherein:
R is selected from H, CH₃, C₂H₅, n-C₃H₇, CH(CH₃)₂, CH₂—C₆H₅, phenyl, 2,4,6-trimethyl-phenyl, 4-chloro-phenyl; 2-pyridyl, 3-pyridyl, 4-pyridyl, 2-thiophenyl, 3-thiophenyl,
and Z is selected from Br, Cl; H; CH₃, C₂H₅, n-C₃H₇, CH(CH₃)₂, n-C₄H₉, CH₂CH(CH₃)₂, C(CH₃)₃, OCH₃, OC₂H₅, O-n-C₃H₇, OCH(CH₃)₂, O-n-C₄H₉, OCH₂CH(CH₃)₂, OC(CH₃)₃; CH═CH₂, CH₂—CH═CH₂, OCH═CH₂, OCH₂—CH═CH₂; C≡CH, CH₂—C≡CH, C₂H₄—C≡CH, CH₂—C≡C—CH₃, OC≡CH, OCH₂—C≡CH, OC₂H₄—C≡CH, OCH₂—C≡C—CH₃; CH₂—C₆H₅, OCH₂—C₆H₅; phenyl, 2,4,6-trimethyl-phenyl, 4-chloro-phenyl, 2-pyridyl, 3-pyridyl, 4-pyridyl; 2-pyrimidyl, 4-pyrimidyl, 5-pyrimidyl; 2-pyrazinyl, 2-furyl, 3-furyl; 2-thiophenyl, 3-thiophenyl; 2-pyrrolyl, 3-pyrrolyl, OC₆H₅, O-4-chloro-phenyl, O-2,4,6-trimethyl-phenyl, O-2-pyridyl, O-3-pyridyl, O-4-pyridyl; O-2-pyrimidyl, O-4-pyrimidyl, O-5-pyrimidyl; O-2-pyrazinyl, O-3-pyrazinyl; O-2-furyl, O-3-furyl; O-2-thiophenyl, O-3-thiophenyl; O-2-pyrrolyl, O-3-pyrrolyl; OH, CH₂OH; CH₂NH₂; CN; COOCH₃, COOC₂H₅, COOPh, COOCH₂Ph.

21. The pharmaceutical composition according to claim 11, wherein the compound has formula Ic 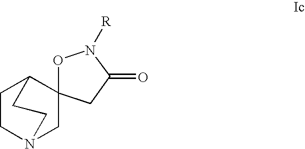
wherein R is selected from H, CH₃, C₂H₅, n-C₃H₇, CH(CH₃)₂, n-C₄H₉, CH₂CH(CH₃)₂, C(CH₃)₃; CH₂—CH═CH₂; CH₂—C≡CH, C₂H₄—C≡CH; phenyl, 2,4,6-trimethyl-phenyl, 4-chloro-phenyl, CH₂—C₆H₅; 2-pyridyl, 3-pyridyl, 4-pyridyl, 2-thiophenyl, 3-thiophenyl.
